(19) United States
(12) Patent Application Publication
(10) Pub. No.: US 2026/0104355 A1
(43) Pub. Date: Apr. 16, 2026

(54) METHOD FOR ONLINE MONITORING OF GEL STRENGTH CHANGES DURING SURIMI THERMAL PROCESSING BASED ON HYPERSPECTRAL IMAGING

(71) Applicant: JIMEI UNIVERSITY, Xiamen (CN)

(72) Inventors: Quansheng CHEN, Xiamen (CN); Yu XIA, Xiamen (CN); Fangling JIANG, Xiamen (CN); Qingmin CHEN, Xiamen (CN); Yi XU, Xiamen (CN); Mingyuan ZHENG, Xiamen (CN)

(21) Appl. No.: 19/400,077

(22) Filed: Nov. 25, 2025

(30) Foreign Application Priority Data
Nov. 26, 2024 (CN) 202411704185.4

(51) Int. Cl. G01N 21/3563; G01N 21/31; G01N 21/359; G01N 33/12

(52) U.S. Cl. CPC G01N 21/3563; G01N 21/314; G01N 21/359; G01N 33/12; G01N 2021/3155; G01N 2021/3572; G01N 2201/126

(57) ABSTRACT

A method for online monitoring of gel strength changes during surimi thermal processing based on hyperspectral imaging includes following steps: raw hyperspectral data of surimi samples during a heating process is acquired, and gel strength of the surimi samples is measured; the raw hyperspectral data is preprocessed; a dataset is constructed based on preprocessed raw hyperspectral data and the gel strength; a partial least squares (PLS) model is trained and validated using the dataset to obtain a gel strength prediction model; and the gel strength prediction model is utilized to monitor changes in gel strength during surimi thermal processing.

### CROSS-REFERENCE TO RELATED APPLICATIONS

This application claims the benefit of priority from Chinese Patent Application No. 202411704185.4, filed on Nov. 26, 2024. The content of the aforementioned application, including any intervening amendments made thereto, is incorporated herein by reference in its entirety.

### TECHNICAL FIELD

This application relates to food quality detection, and more particularly to a method for online monitoring of gel strength changes during surimi thermal processing based on hyperspectral imaging.

### BACKGROUND

In recent years, driven by rapid societal development and abundant fishery resources, surimi products have gained immense popularity among consumers due to their excellent gelling properties, high nutritional value, crisp texture, ready-to-eat convenience, and alignment with contemporary dietary preferences. Currently, surimi raw materials are primarily used in the production of various surimi products such as fish balls, fish cakes, fish rolls, fish sausages, fish tofu, and simulated seafood (such as artificial crab meat, shrimp meat, shark fin, and scallop), which hold substantial economic value. However, surimi is susceptible to various factors during processing, significantly impacting both yield and quality. Consequently, developing methods for rapid, non-destructive, and online monitoring of gel strength during thermal processing is crucial.

In recent years, some non-destructive techniques for monitoring quality changes during food processing include infrared imaging, near-infrared spectroscopy, machine vision, and electronic nose systems. These techniques capture relatively limited sample information. In contrast, hyperspectral imaging can simultaneously acquire spectral data and image information from samples, thereby greatly enriching datasets and enabling simultaneous visual quality analysis. Consequently, research on methods for online monitoring of gel strength changes during surimi thermal processing holds significant practical importance.

### SUMMARY

The objective of this application is to provide a method for online monitoring of gel strength changes during surimi thermal processing based on hyperspectral imaging technology, to address the limitations of existing techniques and to achieve online, rapid, and non-destructive monitoring of gel strength during the heating process of industrial surimi production.

Technical solutions of the present disclosure are described as follows.

This application provides a method for online monitoring of gel strength changes during surimi thermal processing based on hyperspectral imaging, comprising:
acquiring raw hyperspectral data of a surimi sample during a heating process, and measuring a gel strength of the surimi sample;
preprocessing the raw hyperspectral data;
constructing a dataset based on preprocessed raw hyperspectral data and the gel strength;
training and validating a partial least squares (PLS) model using the dataset to obtain a gel strength prediction model; and
monitoring the gel strength changes during the surimi thermal processing by the gel strength prediction model.

In some embodiments, step of “preprocessing the raw hyperspectral data” comprises:
performing black-and-white correction on the raw hyperspectral data, wherein the raw hyperspectral data comprises visible and near-infrared (VNIR) and near-infrared (NIR) dual-band hyperspectral data;
extracting region of interest (ROI) from corrected hyperspectral data;
obtaining mean spectral reflectance of all pixels within the ROI as average spectral data of the surimi sample; and
processing the average spectral data through standard normal variable transformation, multiplicative scattering correction, first-order derivative, and second-order derivative.

In some embodiments, the black-and-white correction on the raw hyperspectral data is performed through a formula expressed as:

$$I = \frac{I 0 - Rb}{Rw - Rb} \times 100 ;$$

wherein I represents the corrected hyperspectral data, I0 represents a dark image, Rb represents an original hyperspectral image, and Rw denotes a white reference image.

In some embodiments, the PLS model is expressed as:

$$Y = XA + B = XZ * C + B = DC + B ;$$

$$Z *= {Z ( {P}^{′} Z )}^{- 1} ;$$

wherein A is a PLS coefficient, B is a residual matrix of Y, D is a score matrix of X, Z is a PLS weight, P and C are loadings of X and Y respectively, Z* is a regression coefficient matrix, X is an independent variable, Y is a dependent variable, and P′ is a transpose of a loading matrix of an independent variable matrix X.

In some embodiments, before training the PLS model using the dataset, the method further comprises: simplifying the PLS model using variable selection algorithms comprising variable combination population analysis (VCPA), VCPA-iterative retaining information variables (VCPA-IRIV), VCPA-genetic algorithm (VCPA-GA), and iterative retaining information variables (IRIV).

In some embodiments, step of “simplifying the PLS model using VCPA, VCPA-IRIV, VCPA-GA, and IRIV algorithms” comprises:
based on the VCPA algorithm, gradually reducing a number of variables using an exponential decreasing function, and creating subsets using binary matrix sampling;
based on the IRIV algorithm, evaluating a usefulness of variables by observing changes in root mean square error of cross-validation (RMSECV) after performing variable addition or variable removal;
based on the VCPA-GA algorithm, reducing a variable space, and optimizing selected variables by simulating natural selection, crossover, and mutation processes to find an optimal variable combination; and
based on the VCPA-IRIV algorithm, first reducing the variable space by VCPA, then performing optimization selection by classifying and iterating the variables by IRIV.

In some embodiments, step of “acquiring the raw hyperspectral data of the surimi sample during the heating process” comprises:
placing a container containing the surimi sample on a moving platform and aligning the container with a spectral acquisition device; and
acquiring complete spectral information of the surimi sample through the spectral acquisition device as the moving platform moves.

The present application has the following beneficial effects.

In the present application, after performing pretreatment on surimi, a water bath heating process is conducted. During thermal processing, hyperspectral data in the VNIR (400-1000 nm) and NIR (900-1700 nm) bands are collected, and the gel strength is measured. The acquired hyperspectral data undergoes black-and-white calibration.

The spectral data of all pixels within the region of interest are collected using environment for visualizing images (ENVI) software, and their average value is taken to represent a single sample. The spectral data are then preprocessed to determine the optimal preprocessing method. The PLS model is constructed using spectral data and gel strength. Variable selection algorithms are applied to obtain the optimal model performance for the VNIR and NIR bands during the thermal processing. Visualization analysis of the gel strength changes in surimi during thermal processing is performed to achieve, in practical applications, online monitoring of gel strength changes during surimi thermal processing and to realize the visualization of gel strength. Therefore, even non-professionals can clearly understand the specific conditions during thermal processing.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to illustrate the technical solutions in the embodiments of the present disclosure or in the prior art more clearly, the drawings needed in the description of embodiments will be briefly introduced below. Obviously, for those of ordinary skill in the art, other drawings can be obtained based on these drawings without exerting creative efforts.

FIG. 1 is a flowchart illustrating a method for online monitoring of gel strength changes during surimi thermal processing based on hyperspectral imaging technology according to an embodiment of the present disclosure;

FIGS. 2a and 2b show feature variable extraction results of four variable selection algorithms according to an embodiment of the present disclosure;

FIGS. 3a and 3b show optimal model parameters according to an embodiment of the present disclosure, where FIG. 3a is a principal component score plot from cross-validation of an optimal PLS model, and FIG. 3b is a result regression plot of the optimal model; and

FIG. 4 shows visualization analysis of the optimal model according to an embodiment of the present disclosure.

### DETAILED DESCRIPTION OF EMBODIMENTS

The technical solutions in the embodiments of the present disclosure will be clearly and completely described below with reference to the accompanying drawings. It should be understood that the described embodiments are merely some embodiments of the present disclosure, and other embodiments obtained by those skilled in the art based on the embodiments of the present disclosure without creative labor fall within the protection scope of the present disclosure.

To make the purpose, features, and advantages of the present disclosure clearer, the disclosure is further detailed below with reference to the accompanying drawings and specific embodiments.

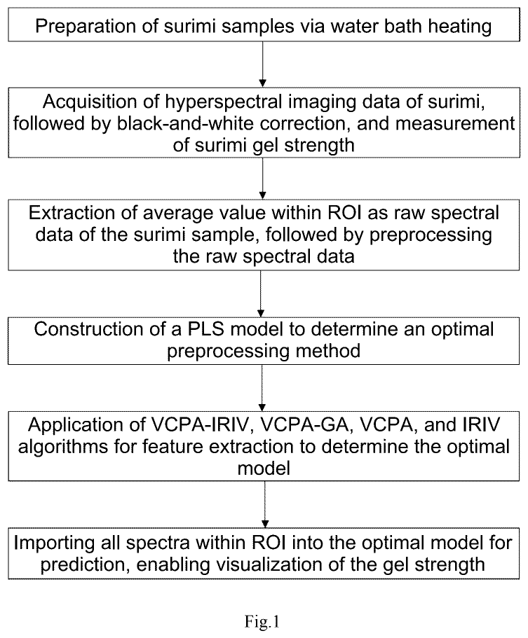

As shown in FIG. 1, this embodiment provides a method for online monitoring of gel strength changes during surimi thermal processing based on hyperspectral imaging technology, including the following steps.

Raw hyperspectral data of surimi samples during the heating process is acquired, and gel strength of the surimi samples is measured.

The raw hyperspectral data is preprocessed.

A dataset is constructed based on the preprocessed raw hyperspectral data and the gel strength.

The PLS model is trained and validated using the dataset to obtain a gel strength prediction model.

The gel strength prediction model is utilized to monitor changes in gel strength during surimi thermal processing.

Specifically, in this embodiment, the method for online monitoring of gel strength changes during surimi thermal processing based on hyperspectral imaging technology included the following steps. A certain amount of frozen silver carp surimi (grade AA) was obtained from Xiamen Anjing. The frozen surimi was thawed and minced in a meat grinder. 2.5% NaCl was added into surimi, and the water content was adjusted to 80%, followed by placing into aluminum containers, heating at 40° C. for 30 minutes and 90° C. for 20 minutes, and taking 30 samples every 5 minutes for a total of 300 samples. Hyperspectral data of the surimi samples were collected, and the gel strength of each surimi sample was measured. Spectral data from the regions of interest in the hyperspectral images of the samples were extracted. A PLS quantitative model was constructed using spectral data and the surimi gel strength. Variable selection algorithms including VCPA, VCPA-IRIV, VCPA-GA, and IRIV were employed to simplify the model and enhance its robustness. This embodiment enabled online, rapid, and non-destructive monitoring of gel strength during the industrial heating process of surimi.

Specifically, simplifying the PLS model refers to reducing the variables used to construct the PLS model, thereby decreasing the computational time of the model. Specifically, these algorithms screen for important variables based on their contribution rate and significance to the model.

Furthermore, step of “the raw hyperspectral data of the surimi samples of the surimi samples during the heating process is acquired” includes placing a container containing the surimi on a moving platform and aligning it with a spectral acquisition device, and acquiring complete spectral information of the surimi through the spectral acquisition device as the moving platform moves.

In this embodiment, first, the surimi was pretreated and then placed in aluminum containers for heating.

Specifically, the frozen surimi used in this embodiment was Grade AA. The process included: thawing the surimi, cutting into small pieces, placing in the meat grinder, and stirring for 3 minutes, adding 2% NaCl, adjusting the water content to 80%, continuing to stir for 3 minutes, and then placing it into the cylindrical aluminum container with a diameter of 2 cm and a height of 2 cm for heating at 40° C. for 30 minutes, followed by 90° C. for 20 minutes.

Next, hyperspectral data of the surimi during the heating process was collected, and the gel strength of each surimi sample was measured, yielding a total of 300 data points.

In this embodiment, dual-band (VNIR and NIR) hyperspectral data were acquired every 5 minutes during the heating process. After data acquisition, the gel strength of the surimi was immediately measured at a test speed of 1 mm/s, a compression distance of 12 mm, and a trigger force of 15 N. The gel strength was calculated as: Gel strength=Breaking force (g) x Breaking distance (cm).

For the hyperspectral image acquisition, the hyperspectral imaging system used in this embodiment includes a hyperspectral imager, a platform control system, and a computer. Specifically, the near-infrared hyperspectral imaging (NIR-HSI, 900-1700 nm) system consists of an imaging spectrometer (N17E, Specim, Finland) with a spectral resolution of 4 nm, a camera with a resolution of 640×512, a camera lens (640miniRaptor, Northern Ireland), two tungsten lamps (LS-150, Wuling Optics, Taiwan, China), a stepper motor-driven moving platform (HSIM-800, Wuling Optics, Taiwan, China), and a computer equipped with software.

The specific method for hyperspectral image acquisition was as follows. The aluminum container containing surimi was placed on the moving platform and aligned with the camera. As the platform moved, the hyperspectral imager captured spectral information across the entire spectral range for a line corresponding to the spatial position of the surimi. The platform then moved the aluminum container to acquire spectral information at other positions of the surimi until the complete spectral information of the sample was obtained. In the VNIR spectral imaging system, the sample stage moved at a speed of 8.3 mm/s with an exposure time of 90 ms and an image resolution of 1604×605 pixels. Since the spectral scanning range spanned 678 effective wavelengths within the 400-1000 nm band, the size of the final acquired three-dimensional data block was 1604×605×678. The NIR spectral imager had a spectral resolution of 4 nm. The camera and lens had a resolution of 640×512. The image resolution was 640×459 pixels. Since the spectral scanning range spanned 678 effective wavelengths within the 400-1000 nm band, the size of the final acquired three-dimensional data block was 640×459×512. The entire acquisition process was conducted inside a dark box. White and black reference images were also collected for subsequent calibration to mitigate ambient light interference on the acquired hyperspectral images.

Furthermore, step of “the raw hyperspectral data is preprocessed” includes the following steps.

Black-and-white correction is performed on the raw hyperspectral data, which includes dual-band (VNIR and NIR) hyperspectral data.

The ROI is extracted from the corrected hyperspectral data.

The mean value of the spectral reflectance of all pixels within the region of interest is obtained as the average spectral data of the surimi sample.

The average spectral data is processed using standard normal variate transformation, multiplicative scatter correction, first-order derivative, and second-order derivative.

Specifically, in this embodiment, the ROI is extracted from the corrected hyperspectral image, and the mean spectral reflectance of all pixels within the ROI is taken as the average spectral data for the surimi sample.

In this embodiment, the black-and-white correction on the hyperspectral data is performed through the following formula expressed as:

$$I = \frac{I 0 - Rb}{Rw - Rb} \times 100.$$

In above formula, I represents the corrected reflectance hyperspectral image expressed in relative reflectance (%), Rb represents the original hyperspectral image, I0 represents the dark image (0% reflectance), and Rw represents the white reference image (100% reflectance).

In a preferred embodiment, a circular region with a radius of 60 pixels was used as the ROI. The boundaries of ROI were determined based on the circular aluminum container holding the surimi. To ensure that the ROI remained a complete circular area when directly utilizing the surimi container for online monitoring, a radius of 60 pixels was set in this embodiment, enabling the online monitoring of gel strength changes during surimi thermal processing. A total of 600 surimi spectral data points were obtained across the two bands.

The obtained average spectral data was preprocessed. Subsequently, the preprocessed average spectral data and the corresponding gel strength values were divided into training and prediction sets in a ratio of 7:3 for subsequent model construction.

In this embodiment, preprocessing methods such as standard normal variable transformation, multiplicative scattering correction, first-order derivative, and second-order derivative were applied to the average spectral data. This yielded hyperspectral data with smoothed spectral information after noise removal.

Due to the light-scattering properties of the surimi sample surface, as well as noise and human-induced interference from the instrument, the aforementioned four preprocessing methods were employed to obtain the preprocessed spectral data. Using the Kennard-Stone (K-S) method, the dataset was then divided into the training set and the prediction set at a 7:3 ratio.

Specifically, in this embodiment, prior to training the PLS model using the dataset, the following steps were also performed. The PLS model was constructed with gel strength and spectral data. The PLS model was simplified using the VCPA, VCPA-IRIV, VCPA-GA, and IRIV algorithms. Visualization analysis was performed using the optimal model.

VCPA is inspired by the “survival of the fittest” principle observed in natural evolution, employing an EDF to gradually reduce the number of variables. Binary matrix sampling is then adopted to ensure equal selection opportunities for each variable, thereby creating various subsets. Model population analysis is employed to identify the subset with the lowest RMSECV.

IRIV is a novel variable selection technique based on a binary matrix transform filter method, evaluating the usefulness of variables by observing the changes in RMSECV after adding or removing variables. Through multiple iterations, IRIV retains both strong and weak informative variables while eliminating non-informative and interfering variables. The retained variables are then reversed to determine the optimal subset.

In VCPA-GA, the variable space is reduced using VCPA, and the final EDF residual variables are then set to 100. The selected variables are further optimized using GA by simulating the processes of natural selection, crossover, and mutation to find the optimal variable combination.

The hybrid VCPA-IRIV strategy combines VCPA and IRIV to handle high-dimensional spectral data. VCPA first reduces the variable space, and then IRIV optimizes the selection by classifying and iterating the variables to eliminate those variables with less information. This hybrid approach leverages the systematic reduction capability of VCPA and the iterative filtering function of IRIV to optimize the variable selection process.

The goal of simplifying the PLS model is to reduce the dimensionality of the X matrix by filtering out important variables (features), thereby making the PLS model more concise, accurate, and robust. When performing stepwise regression using VCPA and its hybrid strategy algorithms, the model optimizes predictive performance by gradually adding or removing independent variables. Cross-validation techniques assist in evaluating the impact of different feature subsets on the model's generalization ability, thereby selecting the most suitable variable subset to simplify the PLS model. Ultimately, the purpose of variable selection is to reduce the number of variables in the dataset, resulting in a smaller input dataset and faster model execution.

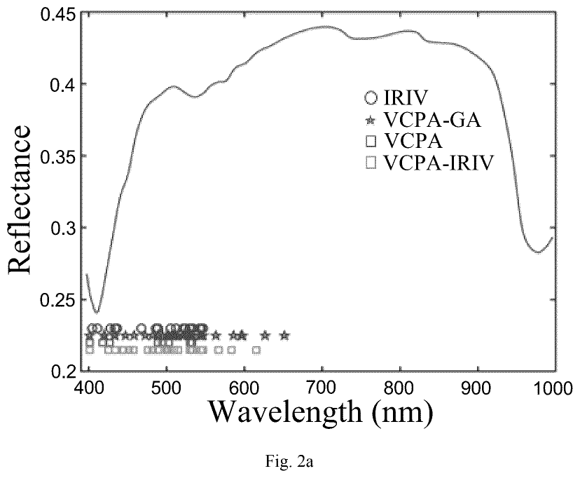

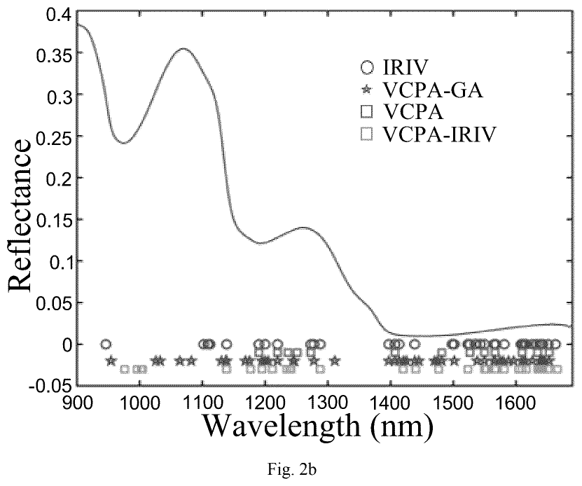

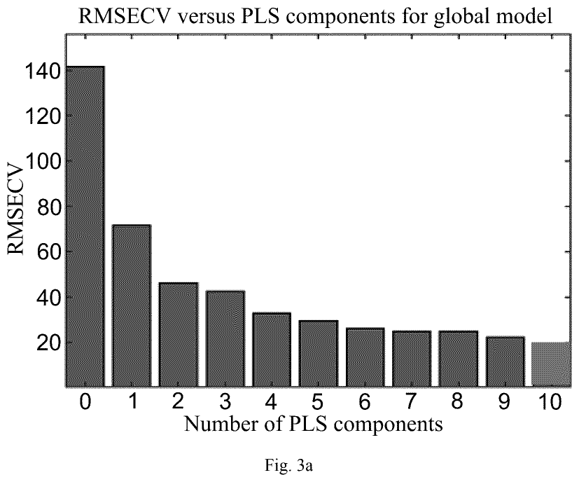

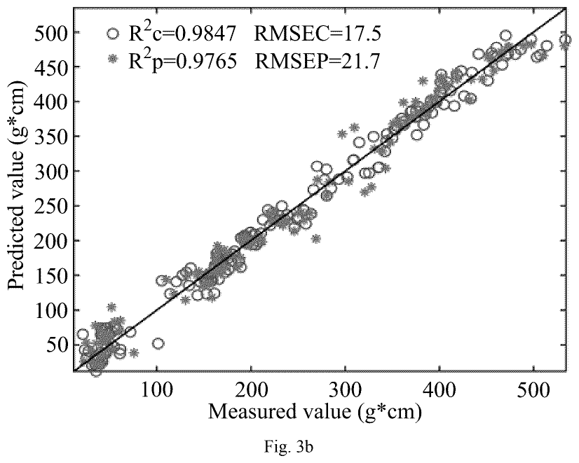

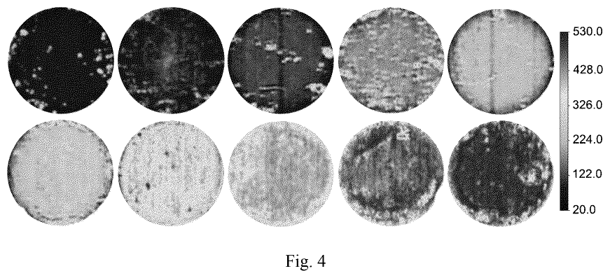

In this implementation, the preprocessed spectral data and the corresponding gel strength of surimi were used for PLS modeling. The optimal preprocessing method was determined, with specific results shown in Table 1. Subsequently, based on the optimally preprocessed PLS model, the VCPA, VCPA-IRIV, VCPA-GA, and IRIV algorithms were applied to simplify the PLS model, and the results were shown in Table 2, with the selected wavelengths illustrated in FIGS. 2a and 2b. The optimal model was depicted in FIGS. 3a and 3b, where FIG. 3a showed the principal component scores for cross-validation of the best PLS model, and FIG. 3b shows the result regression of the best model. Visualization analysis was performed using the optimal model, as shown in FIG. 4.

PLS projected the independent variables X and the dependent variable Y into latent variable space to establish a linear regression model. This process simultaneously considered the covariance information between X and Y to maximize their correlation. PLS decomposed X and Y into several X-scores (D), the PLS model was constructed using the following equations:

$$Y = XA + B = XZ * C + B = DC + B ; and$$

$$Z *= {Z ( {P}^{′} Z )}^{- 1} .$$

Here, A was the PLS coefficient, B was the residual matrix of Y, D was the score matrix of X, Z was the PLS weight, P and C were the loadings of X and Y respectively, and Z* were the regression coefficient matrix. The set of data projected from the spectral data was termed as the orthogonal factors of the “latent variables.” The optimal number of orthogonal factors depended on the prediction error and was typically achieved the lowest value of the predicted residual error sum of squares (PRESS).

TABLE 1
Results of PLS model for two spectral bands
based on different preprocessing methods

| Spectral | Preprocessing | | | | | |
|---|---|---|---|---|---|---|
| range | method | R_{c}² | RMSEC | Rₚ² | RMSEP | RPD |
| VNIR | Raw Spectra | 0.9412 | 34.3 | 0.9182 | 40.5 | 3.51 |
| (400- | Standard normal | 0.9469 | 32.6 | 0.9164 | 40.9 | 3.47 |
| 1000 | variable | | | | | |
| nm) | transformation | | | | | |
| | First-order | 0.9316 | 37.0 | 0.9112 | 42.2 | 3.37 |
| | derivative | | | | | |
| | Second-order | 0.8969 | 45.4 | 0.8695 | 51.1 | 2.78 |
| | derivative | | | | | |
| | Multiplicative | 0.9458 | 32.9 | 0.9142 | 41.5 | 3.43 |
| | scattering | | | | | |
| NIR | Raw Spectra | 0.8750 | 50.4342 | 0.8684 | 50.6728 | 2.77 |
| (900- | Standard normal | 0.9020 | 44.6646 | 0.8954 | 45.1642 | 3.11 |
| 1700 | variable | | | | | |
| nm) | transformation | | | | | |
| | First-order | 0.8839 | 48.6074 | 0.8716 | 50.0507 | 2.80 |
| | derivative | | | | | |
| | Second-order | 0.9134 | 41.9848 | 0.8763 | 49.1249 | 2.86 |
| | derivative | | | | | |
| | Multiplicative | 0.9081 | 43.2497 | 0.8789 | 48.6010 | 2.86 |
| | scattering | | | | | |

VCPA involves two key processes. Firstly, the EDF is designed based on the simple and effective “survival of the fittest” principle from Darwin's theory of natural evolution and is used to determine the number of variables to retain and progressively reduce the variable space. Assuming the EDF is set to run for N iterations, this means it undergoes N runs to iteratively filter the variables. In other words, the variable space is reduced in units of N iterations. In the i-th run of the EDF, the ratio of remaining variables is calculated as shown in formula (1). Secondly, during each EDF run, the BMS strategy provides equal selection opportunities for each variable and to generate different variable combinations, thereby creating a population of subsets for constructing a population of sub-models. Subsequently, model population analysis (MPA) is applied to identify variable subsets with lower RMSECV. The frequency at which each variable appears in the top 10% of the sub-models is calculated. The higher the frequency, the more important the variable.

$${r}_{i} = {e}^{- θ i} \qquad ( 1 )$$

In above formula (1), θ is the constant parameter that controls the EDF curve. e correlates the curvature of the EDF and positively correlates with the descent speed of the curve. It can be calculated under the following conditions: (1) initially, i=0, and all p variables are used for modeling, resulting in r₀=1; (2) At the r_{N}-th iteration, ω variables remain; then r_{N}=ω/p. ω is the final number of variables remaining after N runs. Under the above conditions, θ can be determined by a formula (2):

$$θ = \frac{ln ( \frac{p}{ω} )}{N} . \qquad ( 2 )$$

In the VCPA-based hybrid strategy, VCPA progressively narrows the variable space using EDF, ultimately reducing and optimizing large variable spaces. To address the existing limitations of GA and IRIV in handling large numbers of variables, VCPA is modified and coupled with GA and IRIV to generate the hybrid variable selection strategy. Additionally, this hybrid strategy helps VCPA compensate for its tendency to select too few variables. The VCPA-based hybrid strategy includes the following two steps. Step 1: VCPA is executed to reduce the variable space. For the modified VCPA in this work, w is set to 100 in the EDF step, indicating that 100 variables remain for further optimization by GA and IRIV. Step 2: GA and IRIV are performed to further optimize the remaining 100 variables. These 100 variables are gradually retained by eliminating other variables that contribute little to the EDF over N iterations. The remaining variables span small space and have been optimized, making it easier and more effective for GA and IRIV to select the optimal variable subset.

TABLE 2
Results of PLS model for two spectral bands
based on four variable selection algorithms

| Spectral | Variable | | | | | | |
|---|---|---|---|---|---|---|---|
| range | selection | Variables | R_{c}² | RMSEC | Rₚ² | RMSEP | RPD |
| VNIR | FULL | 678 | 0.9778 | 21.1000 | 0.9679 | 25.4000 | 5.60 |
| (400-1000 | VCPA | 9 | 0.9896 | 20.1794 | 0.9687 | 25.0158 | 5.68 |
| nm) | VCPA-IRIV | 24 | 0.9844 | 17.6835 | 0.9756 | 22.0911 | 6.14 |
| | VCPA-GA | 37 | 0.9847 | 17.5104 | 0.9765 | 21.7059 | 6.55 |
| | IRIV | 18 | 0.9747 | 22.5005 | 0.9689 | 24.9472 | 5.70 |
| NIR (900- | FULL | 477 | 0.9020 | 44.6646 | 0.8954 | 45.1642 | 3.11 |
| 1700 nm) | VCPA | 13 | 0.9206 | 40.1929 | 0.9015 | 43.8356 | 3.20 |
| | VCPA-IRIV | 30 | 0.9397 | 35.0247 | 0.8977 | 44.6758 | 3.14 |
| | VCPA-GA | 45 | 0.9336 | 36.7667 | 0.9047 | 43.1130 | 3.25 |
| | IRIV | 39 | 0.9324 | 37.0968 | 0.9053 | 42.9875 | 3.26 |

The spectral values corresponding to the pixel points in the hyperspectral images of the prepared surimi samples were extracted. The selected spectral values were then input into the optimal model to predict the gel strength information at each pixel point in the surimi hyperspectral images. Finally, through information fusion based on hyperspectral imaging technology, the information distribution map during surimi thermal processing on the plane was reconstructed according to the pixel coordinate information and their corresponding gel strength, as shown in FIG. 4. In the gel strength distribution map, the color changed from blue to red. The redder color indicated the higher gel strength, while the bluer color indicated the lower gel strength.

This embodiment provides the method for online monitoring of gel strength changes during surimi thermal processing, which constructs the PLS regression model using spectral data and gel strength of the surimi during thermal processing, simplifies the model via feature selection methods, and determines the optimal model for visualization analysis. In practical processing applications, no sampling of surimi is required, eliminating contamination and achieving online monitoring.

Described above are merely preferred embodiments of the disclosure, which are not intended to limit the disclosure. It should be understood that any modifications and replacements made by those skilled in the art without departing from the spirit of the disclosure should fall within the scope of the disclosure defined by the appended claims.

What is claimed is:

1. A method for online monitoring of gel strength changes during surimi thermal processing based on hyperspectral imaging, comprising:
acquiring raw hyperspectral data of a surimi sample during a heating process, and measuring a gel strength of the surimi sample;
preprocessing the raw hyperspectral data;
constructing a dataset based on preprocessed raw hyperspectral data and the gel strength;
training and validating a partial least squares (PLS) model using the dataset to obtain a gel strength prediction model;
monitoring the gel strength changes during the surimi thermal processing by the gel strength prediction model;
step of preprocessing the raw hyperspectral data comprises:
performing black-and-white correction on the raw hyperspectral data, wherein the raw hyperspectral data comprises visible and near-infrared (VNIR) and near-infrared (NIR) dual-band hyperspectral data;
extracting region of interest (ROI) from corrected hyperspectral data;
obtaining mean spectral reflectance of all pixels within the ROI as average spectral data of the surimi sample;
processing the average spectral data through standard normal variable transformation, multiplicative scattering correction, first-order derivative, and second-order derivative; and
before training the PLS model using the dataset, the method further comprises:
simplifying the PLS model using variable selection algorithms comprising variable combination population analysis (VCPA), VCPA-iterative retaining information variables (VCPA-IRIV), VCPA-genetic algorithm (VCPA-GA), and iterative retaining information variables (IRIV).

2. The method of claim 1, wherein the black-and-white correction on the raw hyperspectral data is performed through a formula expressed as:
$$I = \frac{I 0 - Rb}{Rw - Rb} \times 100 ;$$
wherein I represents the corrected hyperspectral data, I0 represents a dark image, Rb represents an original hyperspectral image, and Rw denotes a white reference image.

3. The method of claim 1, wherein the PLS model is expressed as:
$$Y = XA + B = XZ * C + B = DC + B ; and$$
$$Z *= {Z ( {P}^{′} Z )}^{- 1} ;$$
wherein A is a PLS coefficient, B is a residual matrix of Y, D is a score matrix of X, Z is a PLS weight, P and C are loadings of X and Y respectively, Z* is a regression coefficient matrix, X is an independent variable, Y is a dependent variable, and P′ is a transpose of a loading matrix of an independent variable matrix X.

4. The method of claim 1, wherein step of simplifying the PLS model using VCPA, VCPA-IRIV, VCPA-GA, and IRIV algorithms comprises:
based on the VCPA algorithm, gradually reducing a number of variables using an exponential decreasing function, and creating subsets using binary matrix sampling;
based on the IRIV algorithm, evaluating a usefulness of variables by observing changes in root mean square error of cross-validation (RMSECV) after performing variable addition or variable removal;
based on the VCPA-GA algorithm, reducing a variable space, and optimizing selected variables by simulating natural selection, crossover, and mutation processes to find an optimal variable combination; and
based on the VCPA-IRIV algorithm, first reducing the variable space by VCPA, then performing optimization selection by classifying and iterating the variables by IRIV.

5. The method of claim 1, wherein step of acquiring the raw hyperspectral data of the surimi sample during the heating process comprises:
placing a container containing the surimi sample on a moving platform and aligning the container with a spectral acquisition device; and
acquiring complete spectral information of the surimi sample through the spectral acquisition device as the moving platform moves.
